(19) United States
(12) Patent Application Publication
(10) Pub. No.: US 2003/0100085 A1
(43) Pub. Date: May 29, 2003

(54) Microbial production of r-phenylacetycarbinol by biotransformation of benzaldehyde by filamentous fungi

(76) Inventors: Michael Breuer, Limburgerhof (DE); Bernhard Hauer, Fussgonheim (DE); Bettina Rosche, Randwick (DE); Peter Rogers, Northwood (AU)

Correspondence Address: Keil & Weikauf, 1350 Connecticut Avenue NW, Washington, DC, 20036, US

(21) Appl. No.: 10/332,035

(22) PCT Filed: Jul. 4, 2001
(86) PCT No.: PCT/EP01/07641

(30) Foreign Application Priority Data
Jul. 5, 2000 (DE) 100-32-058.9

(51) Int. Cl. C12P 7/26; C12N 9/10; C12N 1/16

(52) U.S. Cl. 435/148; 435/254.3; 435/193

(57) ABSTRACT

Process for the production of R-phenylacetylcarbinol by biotransformation of benzaldehyde by filamentous fungi.

The present invention relates to pocess for the production of R-phenylacetylcarbinol (R-PAC) by biotransformation of benzaldehyde by filamentous fungi.

R-phenylacetyl carbinol is an intermediate in the production of the pharmaceutical compound ephedrine and pseudoephedrine and is currently produced via a biotransformation of benzaldehyde by yeast cultures. The biotransformation is catalyzed by the enzyme pyruvate decarboxylase. This catalysis can be conducted using either whole microorganisms (for example Saccharomyces cerevisiae, Candida utilis) or cell free extracts of microorganisms (for example Saccharomyces cerevisiae, Candida utilis, Zymomonas mobilis).

Genes of pyruvate decarboxylases have been isolated from the filamentous fungi Neurospora crassa (Alvarez et al. 1993), Aspergillus parasiticus (Sanchis et al. 1994) and Aspergillus nidulans (Lockington et al. 1997).

In literature the following strains of filamentous fungi are reported to conduct acyloin condensations: in a fermentation of benzaldehyde by Aspergillus niger a diol was detected after treatment with NaBH₄ (Cardillo et al. 1991). Mucor circinelloides is reported for acyloin condensations with acyclic unsaturated aldehydes but not benzaldehyde as substrate (Stumpf and Kieslich 1991).

It was the object of the present invention to provide a process for the microbial production of R-phenylacetylcarbinol by biotransformation of benzaldehyde that with respect to overall yield, enantiomeric purity, stability and safety of microbial catalyst or costs of process should be advantagious over the prior art processes.

A first embodiment of the invention is a process for the production of R-phenylacetylcarbinol by biotransformation of benzaldehyde by filamentous fungi.

Filamentous fungi are classified according to Alexopoulos and Mims (Alexopoulos and Mims, 1979). Preferred for the present invention are filamentous fungi of the subdivisions Ascomycotina, Zygomycotina and Basidiomycotina, especially those selected from the group of Rhizopus, Neurospora, Polyporus, Fusarium, Monilia, Paecilomyces, Mucor. Especially preferred are those of the species Rhizopus javanicus, Neurospora crassa, Polyporus eucalyptorum, Fusarium lateritium, Monilia sitophila, Paecilomyces lilacinus, Mucor rouxii, which are further defined in the experimental section below.

These filamentous fungi are well known to the skilled person and can easily be isolated by known techniques (Onions et al. 1981), or can be obtained from public depositories.

A preselection for suitable filamentous fungi can be made on the capacity of the respective fungus to produce ethanol from sugar (Singh et al., 1992; Skory et al,, 1997).

The biotransformation of benzaldehyde to R-PAC needs the presence of a source of acetaldehyde, which can be acetaldehyde itself or pyruvate. Preferred is the addition of pyruvate, especially in an amount of 1-2, preferred 1.5 mol pyruvate per mol of benzaldehyde.

The filamentous fungi can be used for the biotransformation as whole fungal mycelia or in the form of extracts which contain pyruvate decarboxylase. Extracts means soluble or solubilised forms of enzymes of the fungi. The extracts usually contains enzymes with a higher specific enzymatic activity than the whole fungal mycelia, because of a higher grade of purification.

The enzymes of the extract especially the pyruvate decarboxylase can optionally be stabilised by addition of e.g. natural co-factors of the enzymes, buffers, salts. The pyruvate decarboxylase of the extract can also be used in immobilised form.

The biotransformation process is usually made in water as solvent, preferred in a range of pH between 6.5 and 7.0. The temperature can be varied in a broad range from 0 to 60, preferred from 10 to 40 and especially preferred from 20 to 30° C.

The process can be performed either continuously or as a batch process.

The following examples provide further embodiments and details of the invention.

### EXAMPLE 1

### Determination of Pyruvate Decarboxylase Activity

Pyruvate decarboxylase activity (carboligation activity) was determined by phenylacetyl carbinol formation from the substrates pyruvate and benzaldehyde in 20 min at 25° C. The samples contained 200 μl enzyme solution and 200 μl 2-fold concentrated substrate solution (80 mM benzaldehyde, 200 mM pyruvate, 3 M ethanol, 2 mM thiamine pyrophosphate, 20 mM MgSO₄ in 50 mM MES/KOH pH 7.0). One unit (U) was defined as the amount of enzyme that produces 1 μmol phenylacetyl carbinol per minute. Protein concentrations were estimated according to Bradford. Phenylacetyl carbinol concentrations were determined by HPLC, based on peak areas with reference to phenylacetyl carbinol standards using an Alltima C8 column. For the determination of the phenylacetyl carbinol enantiomers a Chiracel OD column was used.

### EXAMPLE 2

### Biotransformations with Extracts from Fungal Mycelia

Crude extracts of the following strains of filamentous fungi were tested for their capability of transforming benzaldehyde, and pyruvate into phenylacetyl carbinol:

Rhizopus javanicus NRRL 13161

Rhizopus javanicus NRRL 2871

Rhizopus oryzae NRRL 6201

Rhizopus oryzae NRRL 1501

Aspergillus oryzae NRRL 694

Aspergillus tamarii NRRL 429

Neurospora crassa ATCC 9277

Neurospora crassa ATCC 9683

Polyporus eucalyptorum UNSW 805400

Fusarium lateritium UNSW 807100

Fusarium sp. UNSW 871900

Monilia sitophila NRRL1275

Paecilomyces lilacinus NRRL 1746

Mucor rouxii ATCC 44260

NRRL means Northern Regional Research Laboratory (now the National Center For Agricultural Utilization Research) UNSW means University of New South Wales

Strains were grown in cotton stoppered Erlenmeyer-flasks at 30° C. in liquid medium composed of 10 g/l yeast extract, 20 g/l peptone, 90 g/l glucose with an initial pH of 6. Shaking at 230 rpm for 20-70 hours provided oxygen for fast biomass production. The flasks were then covered with parafilm and shaken at 60 rpm for 23-29 hours.

The mycelia were harvested in a Buchner funnel and washed twice with buffer. The frozen mycelium was ground to a powder in a mortar using glass beads as the grinding agent. Breakage buffer was added and the extracts were clarified by centrifugation and adjusted to a set volume. Thus, the crude extracts were about 4-fold concentrated in relation to the culture volume. They were stored in aliquots at −70° C.

Biotransformations were carried out at a scale of 1.2 ml in 2 ml screwed glass vials with 80% v/v crude extract and substrate concentrations of 100 mM benzaldehyde and 150 mM pyruvate in the presence of 20 mM MgSO₄, 1 mM TPP, 1 tablet Complete protease inhibitor (Boehringer)/25 ml and 50 mM MES/KOH pH 7.0.

The vials were rotated vertically at 35 rpm and 22.5° C. After 20 min and after 20 h samples of 300 μl were taken and added to 30 μl 100% [w/v] trichloric acid. After removal of protein by centrifugation, the supernatants were analysed for phenylacetyl carbinol by HPLC.

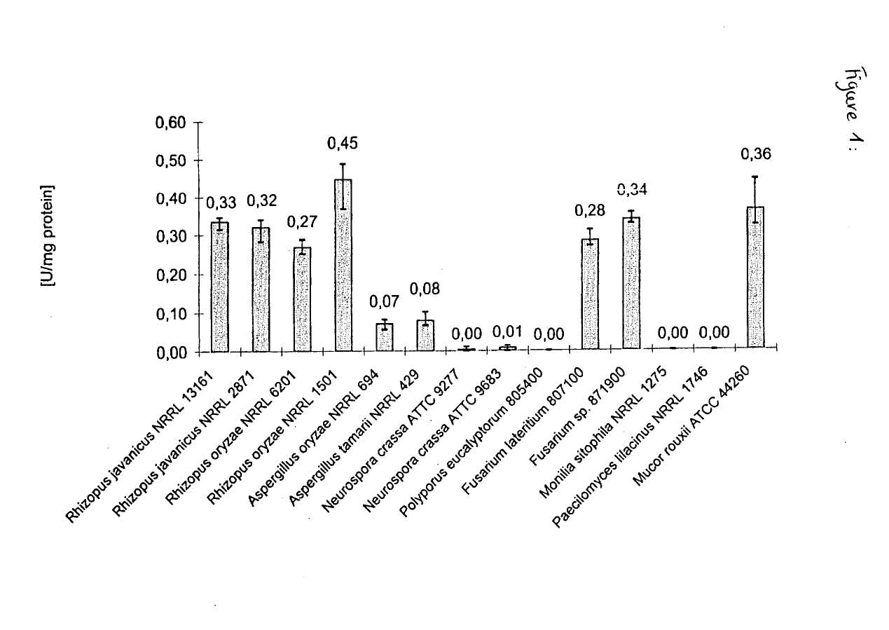

As shown in FIG. 1, highest specific carboligation activities were obtained from the Rhizopus, Fusarium and Mucor with 0.27 to 0.45 U/mg protein The Rhizopus strains also yielded the highest total amount of pyruvate decarboxylase (8.1-15.5 U) that could be recovered from a 20 ml culture.

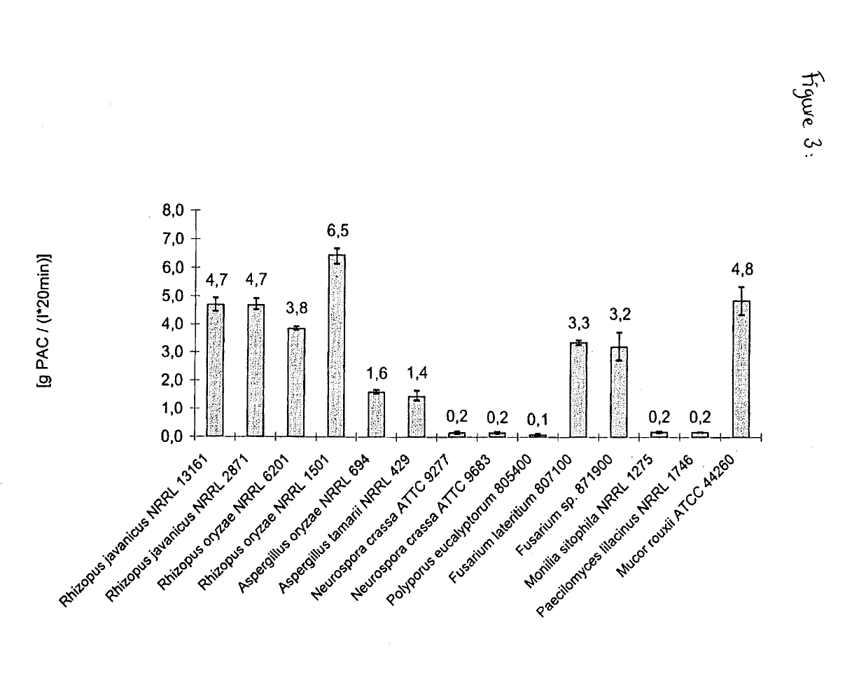

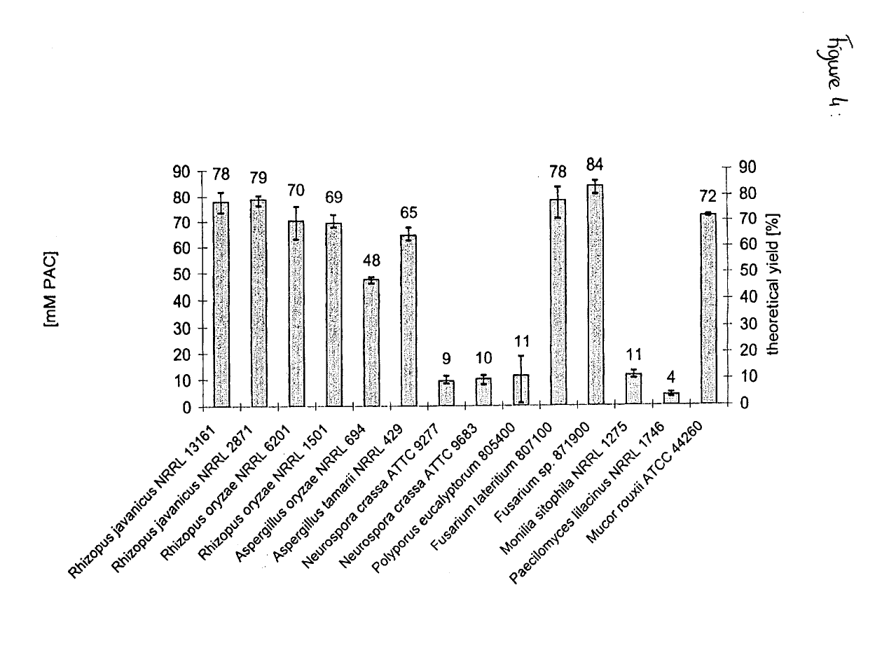

The best initial productivities of 3.8-6.5 g/l phenylacetyl carbinol in 20 minutes were obtained with crude extracts from Rhizopus and Mucor (see FIG. 3). Rhizopus and Fusarium resulted in the highest final phenylacetyl carbinol concentrations of 78-84 mM (11.7-12.6 g/l, see FIG. 4). This was 78-84% of the theoretical yield based on the initial benzaldehyde concentration. These results were obtained without any optimisation of the experimental conditions.

The enantiomeric excess of R-phenylacetyl carbinol from the final biotransformation samples are shown in the following table.

| | enantiomeric excess |
|---|---|
| strain | of R-PAC [%] |
| Rhizopus javanicus NRRL 13161 | 90.4 |
| Rhizopus javanicus NRRL 2871 | 93.0 |
| Rhizopus oryzae NRRL 6201 | 92.9 |
| Rhizopus oryzae NRRL 1501 | 91.4 |
| Aspergillus oryzae NRRL 694 | 92.6 |
| Aspergillus tamarii NRRL 429 | 92.2 |
| Neurospora crassa ATCC 9277 | 73.4 |
| Neurospora crassa ATCC 9683 | not determined |
| Polypous eucalyptorum UNSW 805400 | 98 |
| Fasarium lateritium UNSW 807100 | 91 |
| Fusariuin sp. UNSW 871900 | 92 |
| Monilia sitophila NRRL1275 | 82 |
| Paecilomyces lilacinus NRRL 1746 | 93 |
| Mucor rouxii ATCC 44260 | 91 |

### EXAMPLE 3

### Biotransformations with Whole Fungal Mycelia

The following strains of filamentous fungi were tested for their capability of transforming benzaldehyde into phenylacetyl carbinol using whole mycelia:

Rhizopus javanicus NRRL 13161

Rhizopus javanicus NRRL 2871

Rhizopus oryzae NRRL 6201

Rhizopus oryzae NRRL 1501

Aspergillus oryzae NRRL 694

Aspergillus tamarii NRRL 429

The strains were grown in YEPG medium (90 g/l glucose, 10 g/l yeast extract, 20 g/l peptone, initial pH 6) in cotton stoppered Erlenmeyer flasks at 30° C. The Rhizopus strains were shaken at 230 rpm for 12 hours, the Aspergillus strains for 48 hours. In order to induce pyruvate decarboxylase, the cultures were transferred into sterile screwed glass vials and were left standing at 30° C. for 3.5 h. Gas was produced at a high rate, indicating a high activity of pyruvate decarboxylase.

The culture broth was discarded and an equal amount of YEPG including 100 mM benzaldehyde was added. The cultures were shaken in the screwed glass vials at 30° C. and 230 rpm.

Only 0.2-0.7 mM phenylacetyl carbinol was produced from 100 mM benzaldehyde in 12 hours and the phenylacetyl carbinol concentrations were not increased after further 12 hours. Despite of the low amounts, it is shown, that phenylacetyl carbinol can be produced from benzaldehyde without prior disruption of the mycelia.

### EXAMPLE 4

### Biotransformation of Benzaldehyde by Rhizopus javanicus PDC

The PDC of Rhizopus javanicus was partially purified by acetone precipitation.

Reaction Composition:

0.6-2 M (preferable 2 M) MOPS/KOH, pH 7

20 mM MgSO₄

1 mM TPP

150-600 mM pyruvate (ratio pyruvate/benzaldehyde=1.5)

100-394 mM benzaldehyde

7.2 U/ml PDC carboligase activity

(1 Unit carboligase activity is defined as the amount of enzyme that produces 1 μmol PAC from 40 mM benzaldehyde and 100 mM pyruvate in 1 min at pH 7 and 25° C.)

The reaction was started by adding PDC enzyme. After mixing at 6° C. for 18 hours the reaction was stopped by diluting samples 20-fold with 10% [w/v] trichloroacetic acid. Protein was removed by centrifugation and PAC concentrations were analysed by HPLC.

Results

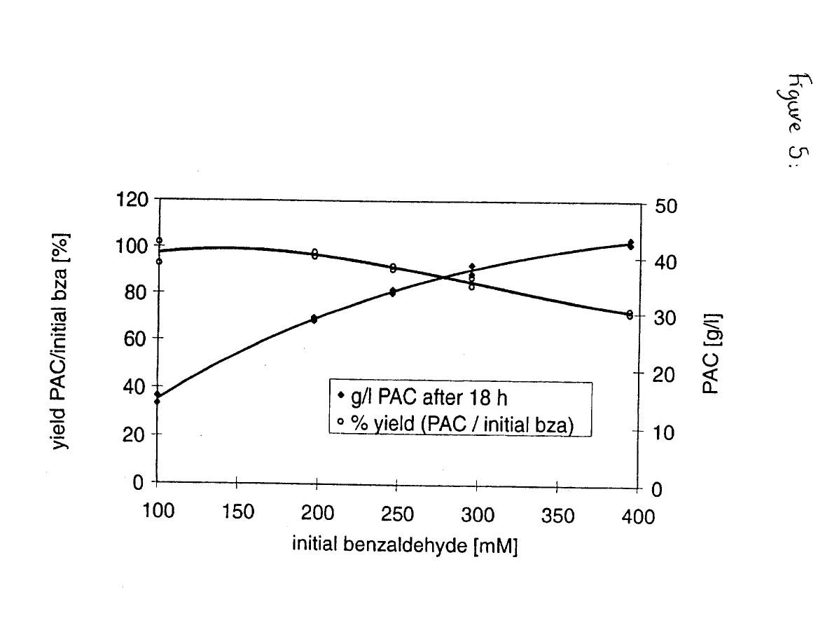

The results are shown in FIG. 5. PAC concentrations of up to 43 g/l were obtained with Rhizopus javanicus PDC. The yields of PAC on initial benzaldehyde were 86% for 295 mM initial benzaldehyde and 73% for 394 mM initial benzaldehyde. The enantiomeric excess (ee-value) was 98.7.

The highest reported PAC concentrations from biotransformations are 28.6 g/l using partially purified PDC from the yeast Candida utilis (Shin and Rogers, 1996; Rogers, Shin and Wang, 1997) and 30.2 g/l in a fermentative process with the yeast Torulopsis (JP 2000-93189A).

References

Alexopoulos, C. J., Mims, C. W.: Introductory Mycology, third edition 1979, John Wiley and Sons, USA

Alvarez, M. E., Rosa, A. L., Temporini, E. D., Wolstenholme, A., Panzetta, G., Patrito, L. Maccioni, H. J. F.: The 59-kDa polypeptide constituent of 8-10-nm cytoplasmic filaments in Neurospora crassa is a pyruvate decarboxylase. Gene 130, 253-258 (1993)

Bradford, M. M.: A rapid and sensitive method for the quantification of microgram quantities of protein utilizing the principle of protein-dye binding. Anlal. Biochem. 72, 248-254 (1976)

Cardillo, R., Servi, S., Tinti, C.: Biotransformation of unsaturated aldehydes by microorganisms with pyruvate decarboxylase activity. Appl. Microbiol. Biotechnol. 36, 300-303 (1991)

Dalboge, H., Lange, L.: Using molecular techniques to identify new microbial biocatalysts. Tibtech 16,265-272 (1998)

Lockington, R. A., Borlace, G. N., Kelly, J. M.: Pyruvate Decarboxylase and anaerobic survival in Aspergillus nidulans. Gene 191, 61-67 (1997)

Onions, A. H. S., Allsopp, D., Eggins, H. O. W.: Smith's Introduction to Industrial Mycology. Seventh edition 1981, Edward Arnold, G B

Sanchis, V., Vinas, I., Roberts, I. N., Jeenes, D. J., Watson, A. J., Archer, D. B.: A pyruvate decarboxylase gene from Aspergillus parasiticus. FEMS Microbiol. Lett. 117, 207-210 (1994)

Shin, H. S. Rogers, P. L.: Production of L-Phenylacetylcarbinol (L-PAC) from benzaldehyde using partially purifiied pyruvate decarboxylase (PDC). Biotech. Bioeng. 49, 52-62 (1996)

Rogers, P. L., Shin H. S., Wang, B.: Biotransformation for L-ephedrin-production. Advances in Biochemical Engineering Biotechnology 56, 33-59 (1997)

Singh, A., Kumar, P. K. R., Schuegerl, K.: Bioconversion of cellulosic materials to ethanol by filamentous fungi. Adv. Biochem. Eng./Biotech. 45, 30-55 (1992)

Skory, C. D., Freer, S. N., Bothast, R. J.: Screening for ethanol-producing filamentous fungi. Biotech. Lett. 19, 203-206 (1997)

Stumpf, B., Kieslich, K.: Acyloin condensation of acyclic unsaturated aldehydes by Mucor species. Appl. Microbiol. Biotechnol. 34, 598-603 (1991)

JP 2000-93189A

FIG. 1 shows specific carboligation activities in crude extracts. The error bars indicate minimum and maximum results from the three cultures per strain.

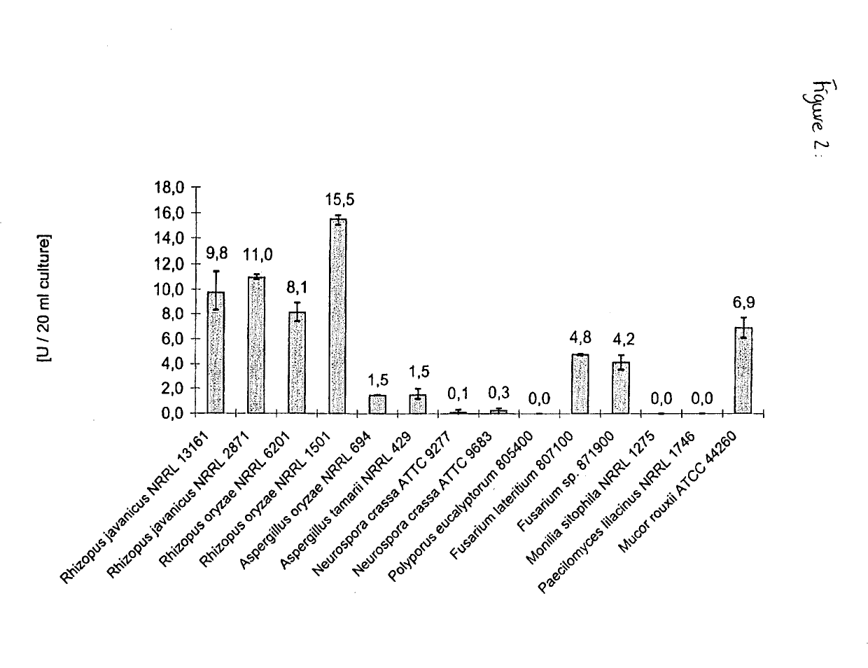

FIG. 2 shows total carboligation activities per flask containing 20 ml culture. The error bars indicate minimum and maximum results from the three cultures per strain.

FIG. 3 shows initial productivity for phenylacetyl carbinol (PAC). The error bars indicate minimum and maximum results from the three cultures per strain.

FIG. 4 shows initial phenylacetyl carbinol (PAC) concentrations and theoretical yields based on initial benzaldehyde concentrations. The error bars indicate minimum and maximum results from the three cultures per strain.

FIG. 5 shows the effect of substrate concentration on PAC production with PDC of Rhizopus javanicus.

1. Process for the production of R-phenylacetylcarbinol by biotransformation of benzaldehyde by filamentous fungi

2. Process according to claim 1 where the filamentous fungi are selected from the group of Rhizopus, Neurospora, Polyporus, Fusarium, Monilia, Paecilomyces, Mucor.

3. Process according to claim 2 where the filamentous fungi are selected from the group of Rhizopus, Fusarium, Mucor.

4. Process according to claim 3 where the filamentous fungi are Rhizopus javanicus or Mucor rouxii.

5. Process according to claim 1-4 where the biotransformation of benzaldehyde is made in the presence of pyruvate.

6. Process according to claim 5 where 1-2 mol pyruvat are added per mol of benzaldehyde.

7. Process according to claim 1-6 where the biotransformation is made by extracts of filamentous fungi.

8. Process according to claim 7 where the extracts contain pyruvate decarboxylase.

9. Process according to claim 8 where the pyruvate decarboxylase is stabilised.
